(19) United States
(12) Patent Application Publication
(10) Pub. No.: US 2017/0355724 A1
(43) Pub. Date: Dec. 14, 2017

(54) ANTHELMINTHIC MACROLIDE SYNTHESIS

(71) Applicant: Norbrook Laboratories Limited, Newry (GB)

(72) Inventors: Andrzej Manikowski, Newry (GB); Karolina Madela, Newry (GB)

(21) Appl. No.: 15/521,061

(22) PCT Filed: Oct. 21, 2015
(86) PCT No.: PCT/GB2015/053152

§ 371 Date: Apr. 21, 2017

(30) Foreign Application Priority Data
Oct. 22, 2014 (GB) 1418783.5

(51) Int. Cl. C07H 1/06; A61K 31/7048; C07H 19/01

(52) U.S. Cl. CPC C07H 1/06; C07H 19/01; A61K 31/7048

(57) ABSTRACT

Disclosed herein is a novel and inventive synthesis of amino-deoxyavermectins, and in particular, the economically significant, anthelminthic macrolide eprinomectin. The synthesis proceeds via reductive amination of an intermediate in which the allylic alcohol of the benzofuran ring is deprotected. Advantageously, the method of the present invention obviates the need for chromatographic purification.

### FIELD OF THE INVENTION

Disclosed herein is a novel and inventive synthesis of 4″-amino-4″-deoxyavermectins and in particular, the economically significant macrolide eprinomectin.

### BACKGROUND TO THE INVENTION

The 4″-amino-4″-deoxyavermectins represent an important class of semi-synthetic avermectins showing improved activity against a range of pests, and parasites relative to their 4″-hydroxy counterparts. The most eminent compounds of this class are emamectin and eprinomectin.

Of particular note, eprinomectin [4″-(epi-acetylamino)-4″-deoxyavermectin], first disclosed in U.S. Pat. No. 4,427,663, is composed of two components; namely, eprinomectin B1a (>90%) and eprinomectin B1b. The B1a (1) and B1b (2) components differ by the presence of an additional methylene unit at the C25 position as illustrated in the below schematic.

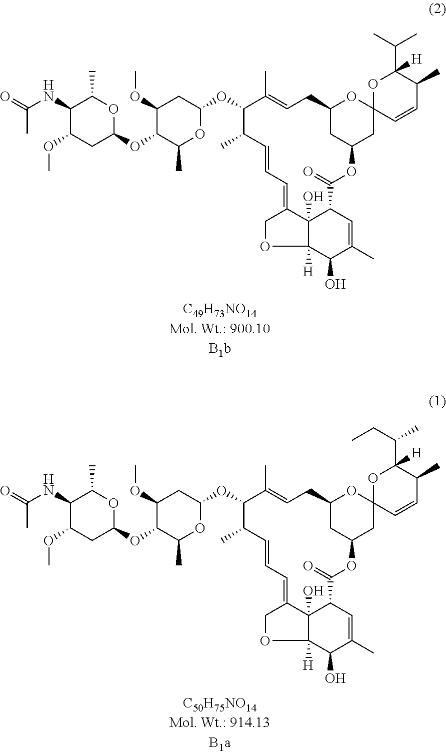 [SMILES: CO[C@H]1C[C@H](O[C@H]2[C@H](C)O[C@@H](O[C@@H]3/C(C)=C/C[C@@H]4C[C@@H](CC5(C=C[C@H](C)[C@@H](C(C)C)O5)O4)OC(=O)[C@@H]4C=C(C)[C@@H](O)[C@H]5OC/C(=C\C=C\[C@@H]3C)[C@]54O)C[C@@H]2OC)O[C@@H](C)[C@@H]1NC(C)=O ; CC[C@H](C)[C@H]1OC2(C=C[C@@H]1C)C[C@@H]1C[C@@H](C/C=C(\C)[C@@H](O[C@H]3C[C@H](OC)[C@@H](O[C@H]4C[C@H](OC)[C@@H](NC(C)=O)[C@H](C)O4)[C@H](C)O3)[C@@H](C)/C=C/C=C3\CO[C@@H]4[C@H](O)C(C)=C[C@@H](C(=O)O1)[C@]34O)O2]

Clinically, eprinomectin has found widespread use as a topical endectocide in cattle. The predilection for use of eprinomectin in cattle is twofold:

firstly, it possesses potent broad-spectrum activity against nematodes, and

secondly, it exhibits an extremely low level of milk/plasma partitioning in comparison to other members of the avermectin/milbemycin families, and results in a zero milk withdrawal times as seen in commercial products such as EPRIZERO by Norbrook Laboratories Limited.

A commercial scale synthesis of eprinomectin devised by Cvetovich in Merck & Co., Inc. is communicated in U.S. Pat. No. 5,362,863. A variant of this synthesis was subsequently reported in Cvetovich et al., J. Org. Chem, 1994, 59, 7704-7708. This synthesis is illustrated in Scheme 1 (vide infra). The authors of this synthesis describe it as the basis of an efficient large scale synthesis on an industrial scale.

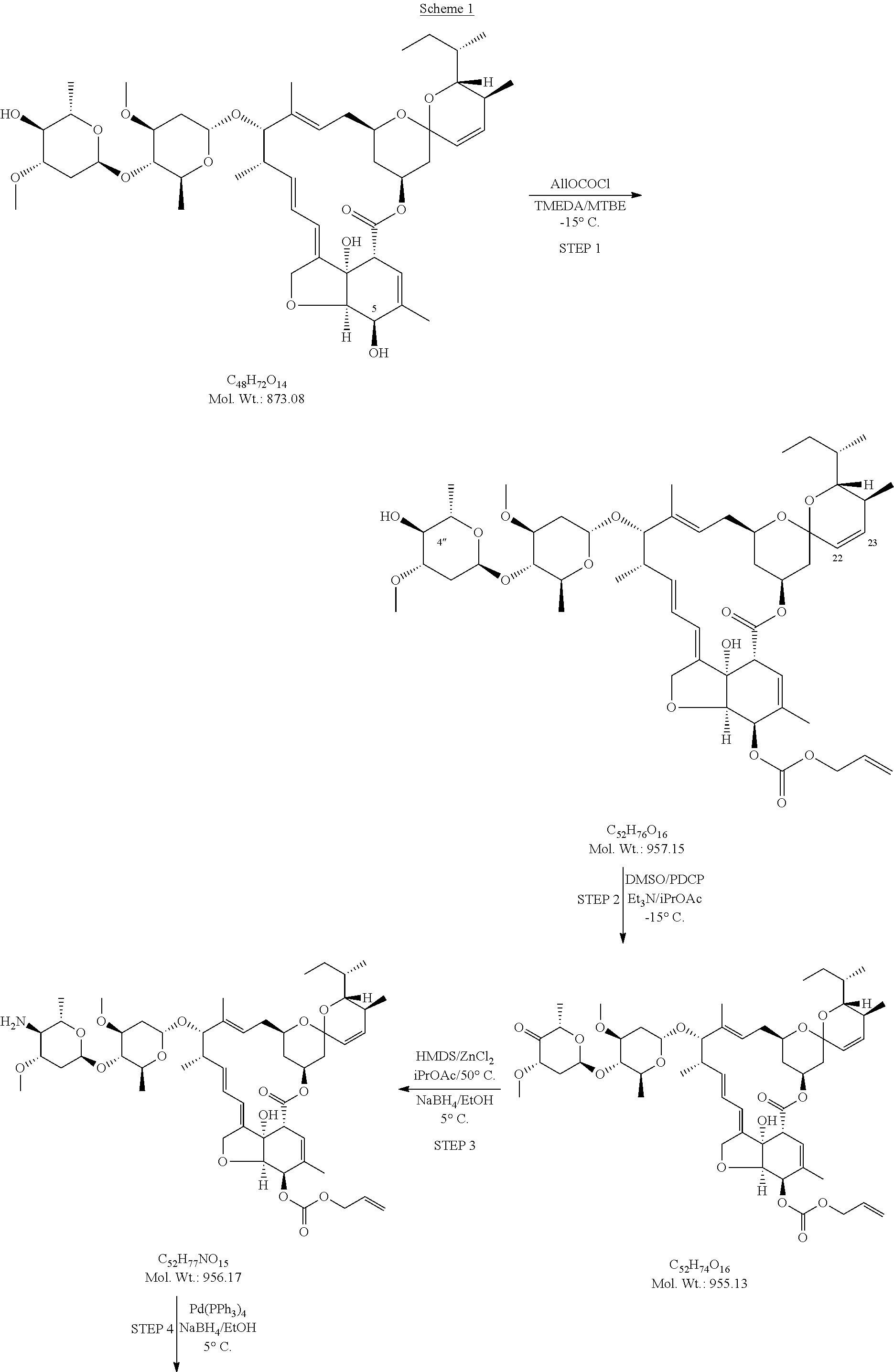

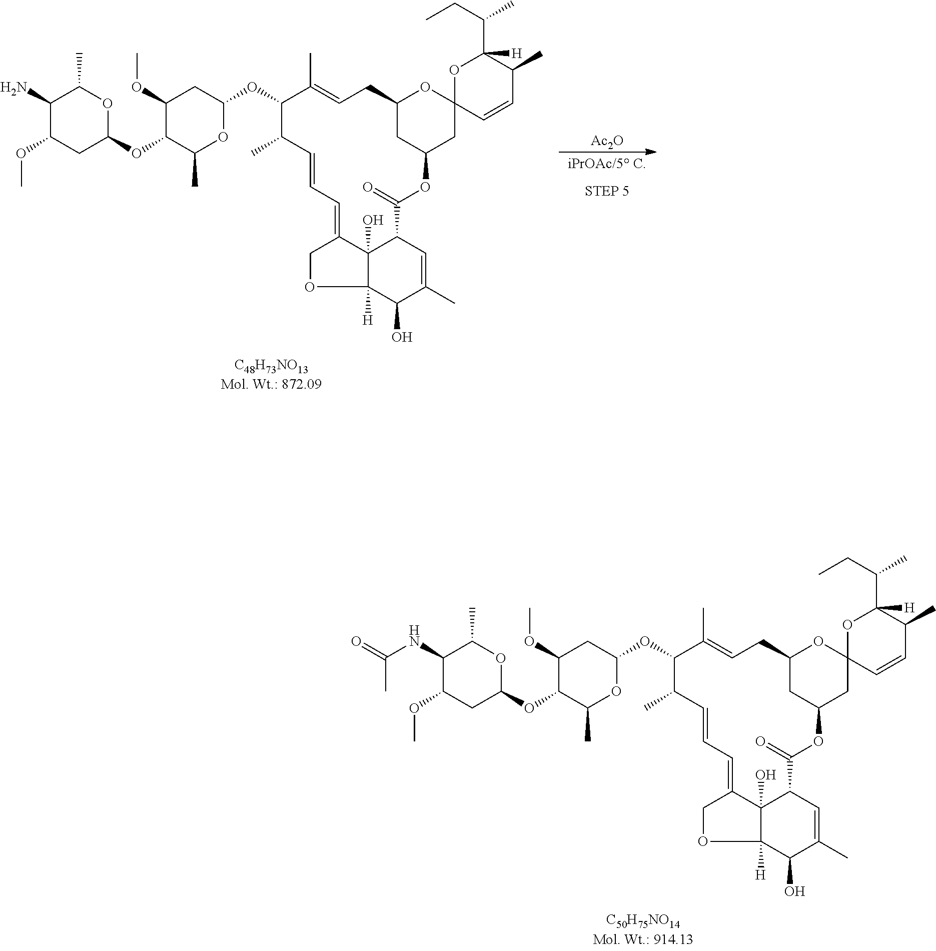 [SMILES: "Ac2O" ; CC[C@H](C)[C@H]1OC2(C=C[C@@H]1C)C[C@@H]1C[C@@H](C/C=C(\C)[C@@H](O[C@H]3C[C@H](OC)[C@@H](O[C@H]4C[C@H](OC)[C@@H](N)[C@H](C)O4)[C@H](C)O3)[C@@H](C)/C=C/C=C3\CO[C@@H]4[C@H](O)C(C)=C[C@@H](C(=O)O1)[C@]34O)O2 ; CC[C@H](C)[C@H]1OC2(C=C[C@@H]1C)C[C@@H]1C[C@@H](C/C=C(\C)[C@@H](O[C@H]3C[C@H](OC)[C@@H](O[C@H]4C[C@H](OC)[C@@H](NC(C)=O)[C@H](C)O4)[C@H](C)O3)[C@@H](C)/C=C/C=C3\CO[C@@H]4[C@H](O)C(C)=C[C@@H](C(=O)O1)[C@]34O)O2]

The synthesis outlined in Scheme 1 comprises the following steps:
1. Reaction of the C5 allylic alcohol with allylchloroformate to yield an allyloxycarbonyl (ALLOC) protected alcohol;
2. Oxidation of the 4″-OH to the corresponding oxo group;
3. Reductive amination of the 4″-oxo group with hexamethyldisilazane (HMDS) to produce the corresponding 4″-amino compound;
4. Removal of the ALLOC protecting group using palladium tetrakis in combination with sodium borohydride/ethanol, and subsequently subjecting the crude material to a filter column/silica plug followed by recrystallization of the material using benzoic acid; and
5. Acetylation of the 4″-amino compound to yield eprinomectin.

However, in reproducing the conditions disclosed in the Cvetovich patent/publication, the present inventors noted that the steps disclosed therein did not reproducibly yield eprinomectin in a sufficiently pure state to the satisfaction of the worldwide regulatory authorities without an additional chromatographic purification step. In particular, by following the Cvetovich patent/publication, the specifications outlined in the U.S. pharmacopoeial monograph for eprinomectin could not be consistently met without subjecting the final material to an additional chromatographic purification step.

The present inventors expended a considerable volume of time analysing the synthetic route proposed in the Cvetovich patent/publication, and isolating/analysing the problematic impurities in order to identify them. The impurities were identified as impurity (3) a 22,23-dihydroeprinomectin derivative and impurity (4), an ethyl carbonate derivative. A further isopropyl carbonate derivative (5) was also observed, but in lower, manageable amounts.

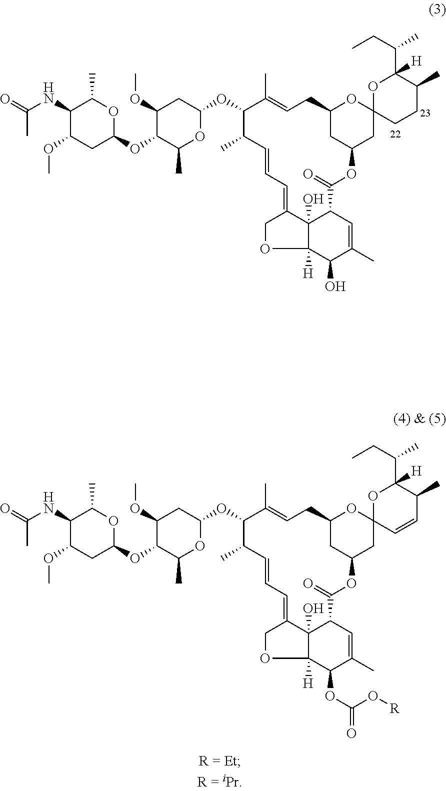

Without the assistance of column chromatography, neither impurity 3 nor 4 could be reduced below acceptable levels such that the batches of eprinomectin reproducibly matched the specifications provided in the U.S. pharmacopoeial monograph. Industrial scale chromatography is undesirable, and should be avoided where possible owing to the costs involved in setting up, and operating such systems.

Accordingly, there remains a need for a synthetic process to eprinomectin and other 4″-amino-4″-deoxyavermectins that does not require a column chromatography purification step.

For the avoidance of any doubt, where the structures and schemes referred to herein present only the major B1a component this is done for ease of understanding. The person skilled in the art will readily understand that the same transformations/conditions are simultaneously applicable to the minor B1b component.

### Definitions

The words “comprises/comprising” and the words “having/including” when used herein with reference to the present invention are used to specify the presence of stated features, integers, steps or components but do not preclude the presence or addition of one or more other features, integers, steps, components or groups thereof.

As used herein, the term ALLOC refers to the allyloxycarbonyl protecting group commonly used to protect alcohols in organic synthesis.

As used herein, the term Cₓ-C_{y} aliphatic refers to linear, branched, saturated and unsaturated hydrocarbon chains comprising Cₓ-C_{y} carbon atoms (and includes Cₓ-C_{y} alkyl, Cₓ-C_{y} alkenyl and Cₓ-C_{y} alkynyl). Similarly, individual references to Cₓ-C_{y} alkyl, Cₓ-C_{y} alkenyl and Cₓ-C_{y} alkynyl include linear and branched Cₓ-C_{y} alkyl, Cₓ-C_{y} alkenyl and Cₓ-C_{y} alkynyl.

The terms, Cₓ-C_{y} cycloalkyl, Cₓ-C_{y} cycloalkenyl, and Cₓ-C_{y} cycloalkynyl include unfused, fused, spirocyclic, polycyclic, hydrocarbon rings.

The term heterocycle refers to cyclic compounds having as ring members atoms of at least two different elements. The cyclic compounds may be monocyclic or polycyclic, and unfused or fused.

As used herein, the term “reductive amination” is utilised in its conventional sense, i.e. conversion of a carbonyl group to an imine, and subsequent reduction of the imine to the amino compound. The imine intermediate may be unsubstituted, mono-substituted, or bis-substituted.

### SUMMARY OF THE INVENTION

The present invention provides for a method of synthesising amino-deoxyavermectins of the general formula (I), or a stereoisomer thereof:

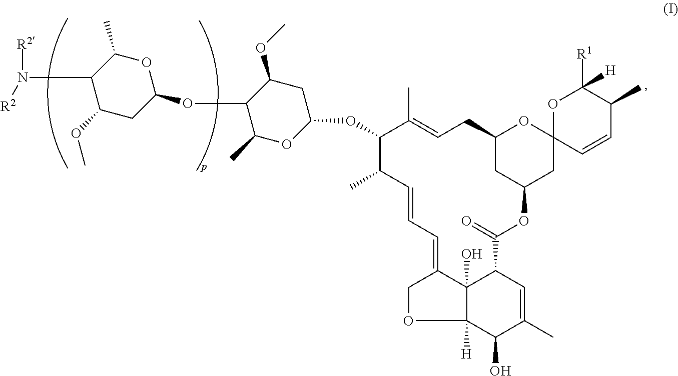

wherein

p is 0 or 1;

R¹ is selected from the group consisting of C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof; and

R² and R^{2′} are the same or different and are selected from the group consisting of H, C₁-C₁₀ acyl, C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof; or

R² and R^{2′} and the nitrogen to which they are attached form a C₃-C₁₀ aliphatic heterocycle.

For example, R¹ is selected from the group consisting of C₁-C₁₀ alkyl, and C₃-C₁₀ cycloalkyl.

R² and R^{2′} are the same or different and are selected from the group consisting of H, C₁-C₁₀ acyl, and C₁-C₁₀ alkyl.

In one embodiment, R¹ is selected from the group consisting of C₁-C₁₀ alkyl, and C₃-C₁₀ cycloalkyl; and R² and R^{2′} are the same or different and are selected from the group consisting of H, C₁-C₁₀ acyl, and C₁-C₁₀ alkyl.

In a preferred embodiment of the present invention, the amino-deoxyavermectins are represented by a structure in which p is 1, R¹ is C₁-C₁₀ alkyl, R²═H, and R^{2′}═C(O)CH₃.

In their investigations, the present inventors attributed the formation of impurities (3) and (4) in the prior art discussed above to step 4 of the route of synthesis illustrated in Scheme 1, supra. In particular, it was noted that:
the 22,23 double bond is labile and is susceptible to reduction in the presence of an excess of Sodium Borohydride (NaBH₄) in ethanol; and
the ALLOC protecting group is vulnerable to a transesterification-type reaction. Repeated exposure to large volumes of ethanol generates ethyl carbonate impurity (3). The ethyloxycarbonyl group cannot be removed/deprotected using palladium tetrakis and impurity (3) is very difficult to separate from eprinomectin.

Accordingly, in a first aspect the present invention provides a method for the synthesis of amino-deoxyavermectins of the general formula (I) supra, or a stereoisomer thereof, the method comprising the step of:
removing an ALLOC protecting group from a compound of the general formula (A), or a stereoisomer thereof to afford the corresponding deprotected compound of the general formula (B), or a stereoisomer thereof:

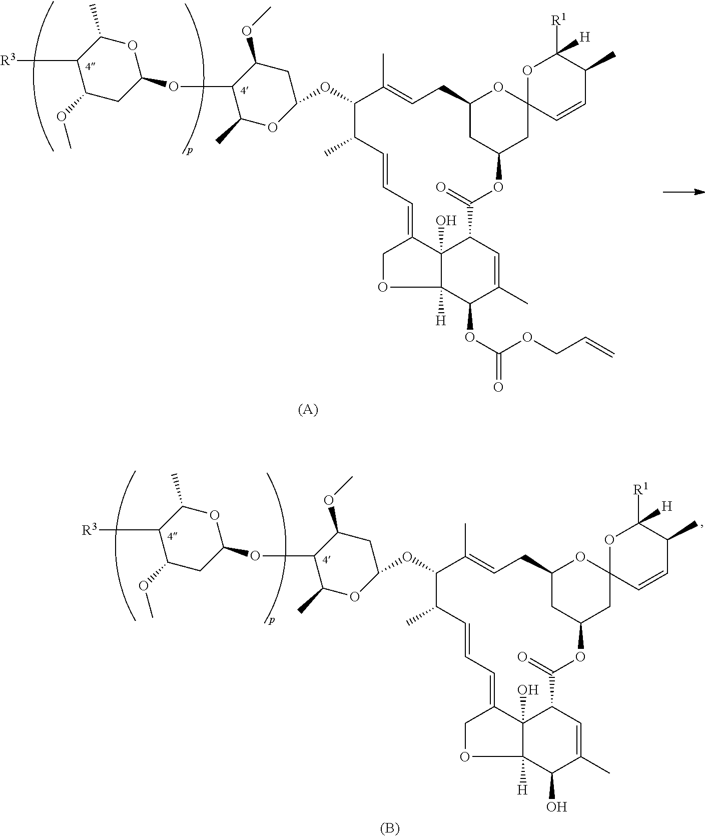

wherein,

p is 0 or 1;

R¹ is selected from the group consisting of C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof; and

R³ is an oxo group, i.e. R³ and the carbon to which it is attached form a C═O moiety.

Desirably, p is 1.

In a preferred embodiment, R¹ may be C₁-C₁₀ alkyl or C₃-C₁₀ cycloalkyl. Preferably, R¹ is C₁-C₅ alkyl. For example, R¹ may be iso-propyl, or sec-butyl.

For example, p may be 1 and R¹ may be C₁-C₁₀ alkyl, such as C₁-C₅ alkyl.

By way of non-limiting theory, in the presence of a palladium catalyst, deprotection of the ALLOC protecting group results in the liberation of an electrophilic, reactive π-allylpalladium complex, vide infra. A sacrificial nucleophile is required to react with the allylpalladium complex to prevent unwanted allylation of the target molecule.

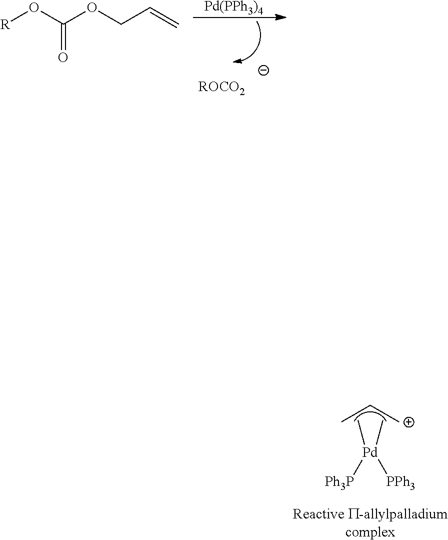

In the prior art, removal of the ALLOC protecting group is completed after the reductive amination step; see step 4 of Scheme 1. Consequently, the 4″-amino group generated competes with the sacrificial nucleophile to result in an N-allylated impurity. Weak sacrificial nucleophiles such as formic acid result in significant quantities of the N-allylated impurity. The present inventors have found that whilst a stronger, reducing combination of sodium borohydride in ethanol results in complete elimination of the N-allylated impurity, this combination of reagents also results in unwanted reduction of the 22,23-double bond.

Advantageously, removal of the ALLOC group from intermediate (A), i.e. before generation of the 4″-amino group facilitates trapping of the π-allylpalladium complex using a weak nucleophile. The reducing conditions caused by the sodium borohydride/ethanol reagents can be avoided in this step, thereby preventing undesired reduction of the 22,23-double bond. Further advantageously, no competing allylation of the target molecule is observed as intermediate (B) is absent a nucleophilic amino group.

Further advantageously, by eliminating the use of sodium borohydride/EtOH in the ALLOC removal step the concentration of ethyl carbonate impurity (4) also decreased significantly in the end product.

The ALLOC protecting group may be removed in the presence of a palladium catalyst, and a nucleophilic scavenger. The nucleophilic scavenger may be selected from the group consisting of methanol, ammonium formate, formic acid, acetic acid, sodium acetate, p-toluenesulfinate, ammonium acetate, n-butylamine, diethylamine, pyridine and combinations thereof. In a preferred embodiment, the nucleophilic scavenger comprises acetic acid, and sodium acetate. The preferred solvent of the ALLOC removal step is a C₁-C₁₀ alkyl acetate, for example, iso-propyl acetate.

The method of the present invention may further comprise the step of subjecting the oxo group of a compound of the formula (B), or a stereoisomer thereof to a reductive amination protocol to afford the corresponding amino compound (C), or a stereoisomer thereof,

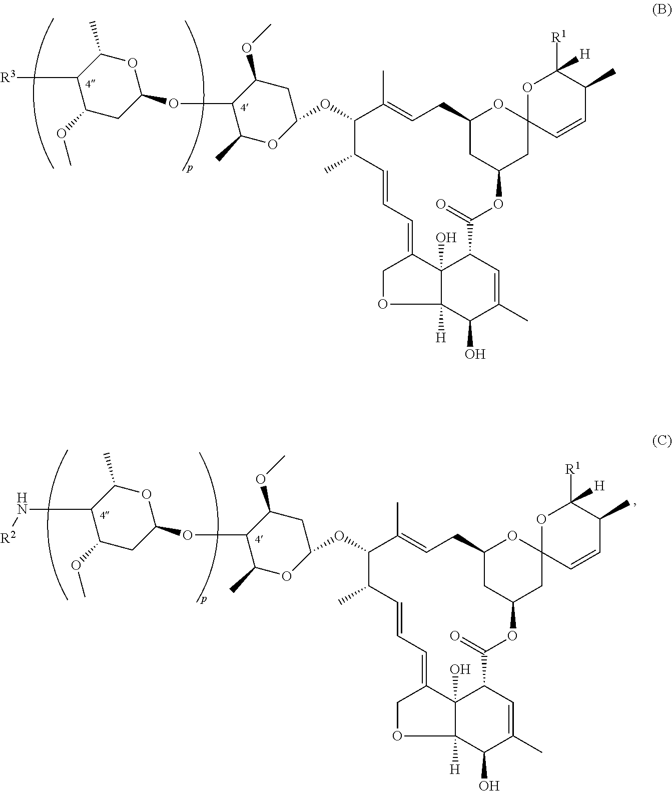

wherein

p is 0 or 1;

R¹ is selected from the group consisting of C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof;

R² is selected from the group consisting of H, C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof; and

R³ is an oxo group, i.e. R³ and the carbon to which it is attached form a C═O moiety.

Preferably p is 1.

R¹ may be C₁-C₁₀ alkyl or C₃-C₁₀ cycloalkyl. Preferably, R¹ is C₁-C₅ alkyl. For example, R¹ may be iso-propyl, or sec-butyl.

R² may be selected from the group consisting of H, and C₁-C₁₀ alkyl.

For example, p may be 1, R¹ may be C₁-C₁₀ alkyl and R² may be selected from the group consisting of H, and C₁-C₁₀ alkyl. Desirably, p is 1, R¹ is C₁-C₅ alkyl and R² is H.

Desirably, the reductive amination protocol is carried out with an amine of the alkyl disilazane class. Such alkyl disilazane compounds may be represented by the general formula (D):

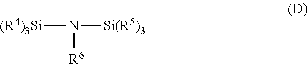

wherein

R⁴, and R⁵ are the same or different and are selected from the group consisting of C₁-C₁₀ alkyl, C₂-C₁₀ alkenyl, and C₂-C₁₀ alkynyl; and

R⁶ is selected from the group consisting of H, C₁-C₁₀ alkyl, C₂-C₁₀ alkenyl, and C₂-C₁₀ alkynyl.

For example, the amine may be selected from the group consisting of hexamethyldisilazane (HDMS), and heptamethyldisilazane (HpDMS).

The reductive amination protocol may be carried out using an amine of the alkyl disilazane class in the presence of sodium borohydride and ethanol. The preferred solvent of the reductive amination step is a C₁-C₁₀ alkyl acetate, for example, iso-propyl acetate.

With further reference to the method of the present invention, when

p is 1,

R¹ is selected from the group consisting of C₁-C₁₀ alkyl, C₁-C₁₀ alkenyl, C₁-C₁₀ alkynyl, C₃-C₁₀ cycloalkyl, C₃-C₁₀ cycloalkenyl, C₃-C₁₀ cycloalkynyl, and combinations thereof; and

R² is H,

the method of the present invention may further comprise the step of acylating a compound of the general formula (C′), or a stereoisomer thereof to produce a compound of the general formula (E), or a stereoisomer thereof.

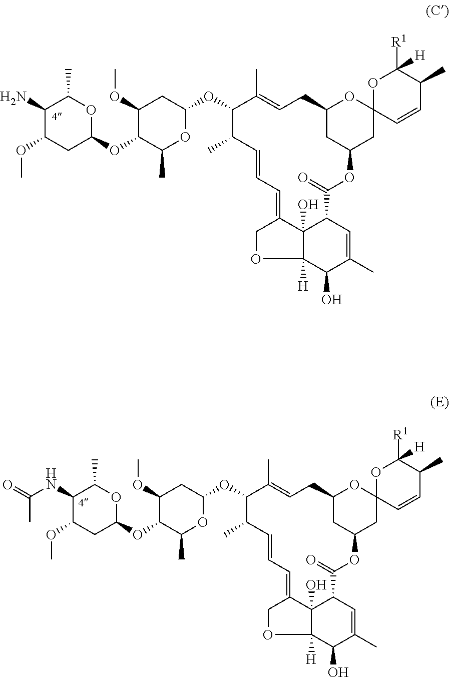

Preferably, R¹ is C₁-C₁₀ alkyl, such as C₁-C₅ alkyl. For example, R¹ may be iso-propyl, or sec-butyl.

In a preferred embodiment of the present invention, compound (E) represents eprinomectin, i.e. wherein R¹ is iso-propyl, or sec-butyl as illustrated below.

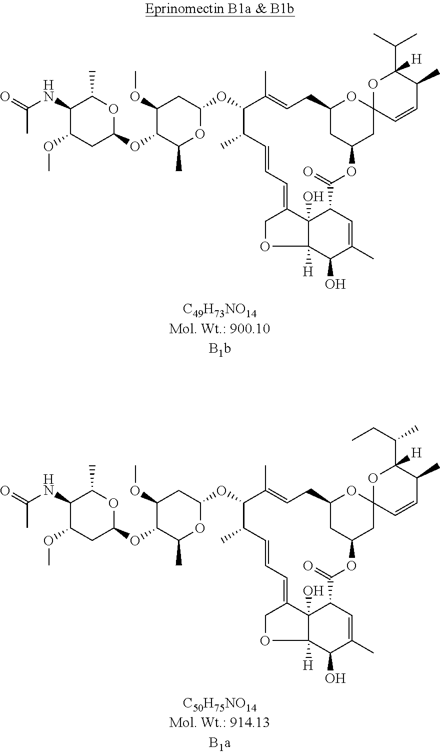 [SMILES: CO[C@H]1C[C@H](O[C@H]2[C@H](C)O[C@@H](O[C@@H]3/C(C)=C/C[C@@H]4C[C@@H](CC5(C=C[C@H](C)[C@@H](C(C)C)O5)O4)OC(=O)[C@@H]4C=C(C)[C@@H](O)[C@H]5OC/C(=C\C=C\[C@@H]3C)[C@]54O)C[C@@H]2OC)O[C@@H](C)[C@@H]1NC(C)=O ; CC[C@H](C)[C@H]1OC2(C=C[C@@H]1C)C[C@@H]1C[C@@H](C/C=C(\C)[C@@H](O[C@H]3C[C@H](OC)[C@@H](O[C@H]4C[C@H](OC)[C@@H](NC(C)=O)[C@H](C)O4)[C@H](C)O3)[C@@H](C)/C=C/C=C3\CO[C@@H]4[C@H](O)C(C)=C[C@@H](C(=O)O1)[C@]34O)O2]

The step of acylating a compound of the general formula (C′) to produce a compound of the general formula (E) may be done with acetic anhydride. The preferred solvent for the acylation step is a C₁-C₁₀ alkyl acetate, for example, iso-propyl acetate.

The method of the present invention may further comprise the step of recrystallizing a compound of the general formula (E), or a stereoisomer thereof from acetonitrile.

In a further aspect, the present invention provides for a molecule of the general formula (II), or a stereoisomer thereof:

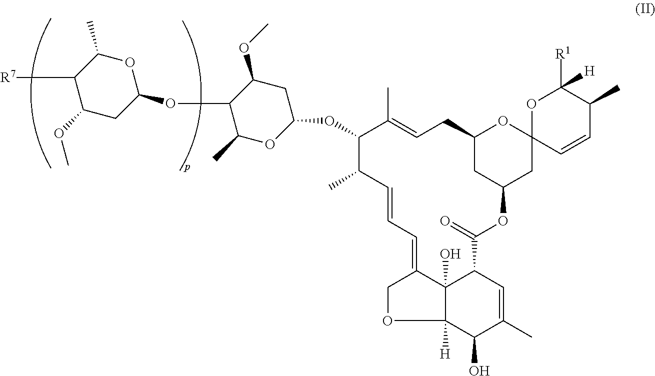

wherein p is 0 or 1;

R¹ is selected from the group consisting of C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof;

R⁷ and the carbon atom to which it is attached form a C═N(R⁸)(R¹⁰)q moiety;

q is 0 or 1;

R⁸ is selected from the group consisting of H, and Si(R⁹)₃;

R⁹ is selected from the group consisting of C₁-C₁₀ alkyl, C₂-C₁₀ alkenyl, and C₂-C₁₀ alkynyl; and

R¹⁰ is selected from the group consisting of H, C₁-C₁₀ alkyl, C₂-C₁₀ alkenyl, C₂-C₁₀ alkynyl, and Si(R⁹)₃,

such that, when q=0, R⁸ is Si(R⁹)₃.

Advantageously, the novel molecule of the general formula (II) is an intermediate in the inventive process disclosed herein. In particular, the novel intermediate (II) is formed during the reductive amination protocol with an alkyl disilazane outlined supra following removal of the ALLOC group.

In a preferred embodiment, p may be 1.

R¹ may be selected from the group consisting of C₁-C₁₀ alkyl, C₁-C₁₀ alkenyl, and C₁-C₁₀ alkynyl. Preferably, R¹ is C₁-C₅ alkyl. For example, R¹ may be iso-propyl, or sec-butyl.

R⁹ may be C₁-C₁₀ alkyl. Preferably, R⁹ is C₁-C₅ alkyl. For example, R⁹ may be methyl.

R¹⁰ may be selected from the group consisting of H, and Si(R⁹)₃.

In a preferred embodiment, p is 1, q is 0, R¹ is C₁-C₅ alkyl, R⁸ is Si(R⁹)₃, and R⁹ is C₁-C₅ alkyl, i.e.

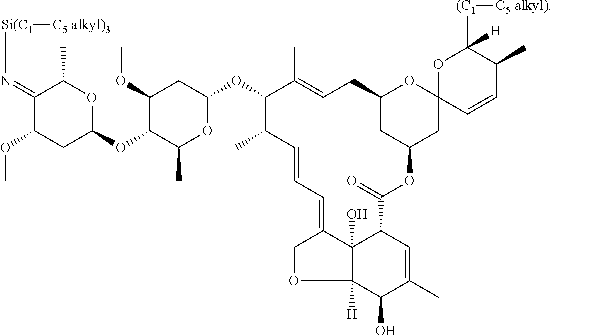

The structures disclosed herein are presented in terms of defined stereochemistry. However, the invention is not to be considered limiting in this regard. In particular, the method of the present invention is equally applicable to the different stereoisomers (diastereomers and enantiomers) of the compounds disclosed herein.

Where suitable, it will be appreciated that all optional and/or preferred features of one embodiment of the invention may be combined with optional and/or preferred features of another/other embodiment(s) of the invention.

### BRIEF DESCRIPTION OF THE DRAWINGS

Additional features and advantages of the present invention are described in, and will be apparent from, the detailed description of the invention and from the drawings in which:

FIG. 1 illustrates a schematic of a synthesis of eprinomectin according to the method of the present invention.

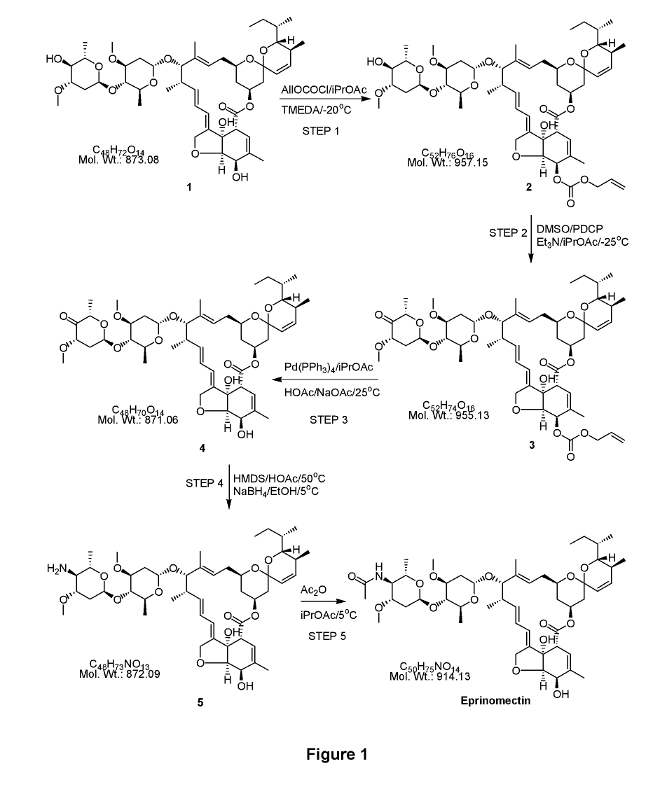

### DETAILED DESCRIPTION OF THE INVENTION

It should be readily apparent to one of ordinary skill in the art that the examples disclosed herein below represent generalised examples only, and that other arrangements and methods capable of reproducing the invention are possible and are embraced by the present invention.

### Step 1. Protection with Allyl Chloroformate

### 5-O-(Allyloxycarbonyl)avermectin B1 (2)

Avermectin (1) (240 g, 0.275 mol) was dissolved in dry iPrOAc (900 mL) and cooled down to 0° C. N,N,N′,N′-Tetramethylethylenediamine (TMEDA) (41.0 mL, 1 eq) was added and a reaction mass was cooled down to −25° C. A solution of allyl chloroformate (36.1 ml, 1.25 eq) in iPrOAc (120 mL) was added drop wise maintaining internal temperature −25 to −20° C. The reaction was stirred for 1 hour and quenched with water (480 mL). The layers were separated and organic layer was washed with water (480 mL). The combined aqueous layers were washed with iPrOAc (2×120 mL) and all organic layers were pooled together, distilled off at <50° C. under vacuum to half of volume, and used directly in the next step. HPLC assay: 85.5%, yield assay: 90%.

### Step 2. Oxidation

### 4″-Oxo-5-O-(allyloxycarbonyl)avermectin B1 (3)

To a solution of 2 (approx. 237 g, 0.247 mol) in iPrOAc (960 mL) were added triethylamine (240 mL, 6.95 eq) and DMSO (108 mL, 6.15 eq). The reaction mass was cooled down to −25° C. and a solution of phenyl dichlorophosphate (96.5 mL, 2.56 eq) in iPrOAc (180 mL) was added drop wise, while maintaining internal temperature between −25 and −20° C. The reaction mixture was stirred for an additional 1 hour and was quenched with water (480 mL). The layers were separated and the organic layer was washed with water (480 mL). The combined aqueous layers were washed with iPrOAc (2×120 mL) and all organic layers were pooled together, distilled off at <50° C. under vacuum to half of volume and used directly in the next step. HPLC assay: 87.24%, yield assay: 80%.

### Step 3. Deprotection

### 4″-Oxo-avermectin B1 (4)

To a solution of 3 (approx. 189 g, 0.198 mol) in iPrOAc (960 mL) was added acetic acid (15.7 mL, 1.31 eq), sodium acetate (45 g, 2.75 eq) and tetrakis triphenylphosphine palladium (0) (7.2 g, 0.031 eq). The reaction mass was stirred at 20-25° C. for 4 hours or until completion of the reaction. 1% NaOH solution (960 mL) and activated charcoal (24 g) were added and the reaction mixture was stirred for 15 min at 20-25° C. Charcoal was filtered off, washed with iPrOAc (2×240 mL), and the layers were separated. The organic layer was washed with a mixture of water (240 mL) and brine (120 mL), and the combined aquatic layers were washed with iPrOAc (2×120 mL). Pooled together organic layers were distilled off to half of volume at <50° C. under vacuum and used directly in the next step. HPLC assay: 80.05%, yield assay: 90%.

### Step 4. Reductive Amination

### 4″-epi-amino-4″-deoxoavermectin B1 (5)

To a solution of 4 (approx. 155 g, 0.178 mol) in iPrOAc (960 mL) were added HMDS (180 mL, 4.75 eq) and AcOH (18.5 mL, 1.8 eq) and all was stirred for 5 hours at 48-52° C. After cooling down to 5° C. ethanol (60 mL) was poured followed by drop wise addition of a precooled to 5° C. solution of sodium borohydride in ethanol (7.6 g, 1.1 eq in 190 mL of ethanol). The reaction mixture was next warmed up to 25° C., stirred for 3 hours and quenched at 5° C. with AcOH (72.0 mL). 5% NaOH solution (960 mL) and activated charcoal (24 g) were added and all was stirred at 20-25° C. for 15 min. Charcoal was filtered off, washed with iPrOAc (2×120 mL) and the layers were separated. The organic layer was washed with 2.5% NaOH (480 mL), and both aquatic layers were combined and washed with iPrOAc (2×120 mL). All organic layers were pooled together and treated with heptane to get a 1:1 iPrOAc:heptane mixture. Product was washed out with a solution of 1% HCl and EtOH (3:1, 2×600 mL) and stirred 1 hour at room temperature. Addition of 5M NaOH brought pH to 9 and product was extracted with iPrOAc (2×600 mL). The combined organic layers were distilled off to half of volume at <50° C. under vacuum and used directly in the next step. HPLC assay: 73.51%, yield assay: 85%.

### Step 5. Acetylation

### 4″-epi-Acetylamino-4″-deoxoavermectin B1 (Eprinomectin)

To a solution of 5 (approx. 132 g, 0.151 mol) cooled down to 5° C. was added acetic anhydride (36 mL, 7.62 eq). The reaction mass was stirred for 1 hour at <5° C. and saturated NaHCO₃ solution (600 mL) was added. The mixture was stirred for 15 min and the layers were separated. The organic layer was washed with brine (600 mL), treated with charcoal (24 g) and stirred for 30 min. Charcoal was filtered off, washed with iPrOAc (2×120 mL), and the combined organic phases were concentrated to half of volume at <50° C. under vacuum (HPLC assay: 74.98%, yield assay: 99%). iPrOAc was exchanged to acetonitrile by three times co-distillation with acetonitrile (3×720 mL) at <50° C. under vacuum to get a dense yellowish suspension. After addition of fresh acetonitrile (240 mL), stirred for 1 hour at room temperature, and for an additional 2 hours at 0° C., the suspension was filtered off, washed with cold acetonitrile (2×120 mL), and dried under vacuum at 40° C. to afford 110 g of crude Eprionomectin. HPLC assay: 97.14%, yield: 87%.

### Crystallization of Eprinomectin

110 g of crude Eprinomectin was dissolved in acetonitrile (1200 mL) under reflux, cooled down to room temperature, stirred for 1 hour and next for 2 hours at 0° C. A white suspension was filtered off, washed with cold acetonitrile (2×100 ml) and dried at 40° C. under vacuum to afford 95 g of Eprinomectin (82% yield). HPLC assay: 98.52%.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A method of synthesising amino-deoxyavermectins of the general formula (I), or a stereoisomer thereof: 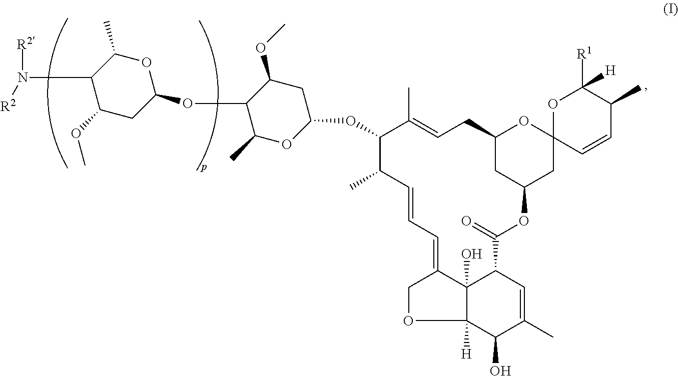
wherein
p is 0 or 1;
R¹ is selected from the group consisting of C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof; and
R² and R^{2′} are the same or different and are selected from the group consisting of H, C₁-C₁₀ acyl, C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof; or
R² and R^{2′} and the nitrogen to which they are attached form a C₃-C₁₀ aliphatic heterocycle,
the method comprising the step of:
removing an ALLOC protecting group from a compound of the general formula (A), or a stereoisomer thereof to afford the corresponding deprotected compound of the general formula (B), or a stereoisomer thereof: 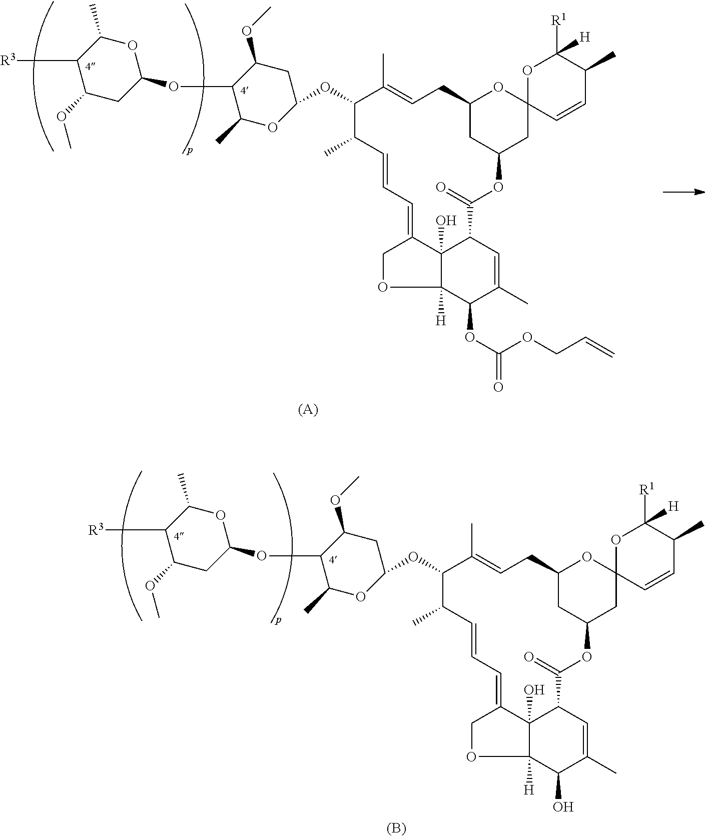
wherein,
p is 0 or 1;
R¹ is selected from the group consisting of C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof; and
R³ is an oxo group.

2. The method of claim 1, further comprising the step of subjecting the oxo group of a compound of the formula (B), or a stereoisomer thereof to a reductive amination protocol to afford the corresponding amino compound (C), or a stereoisomer thereof, 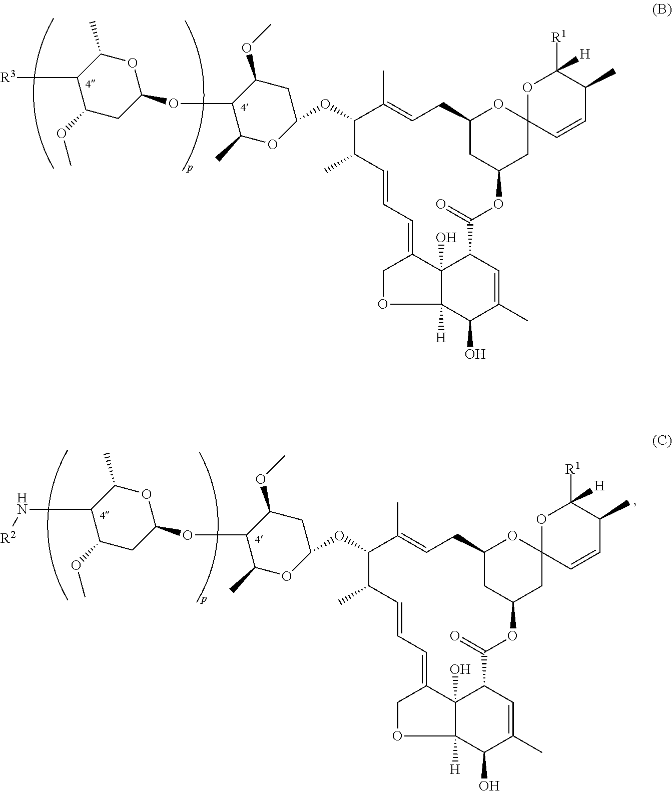
wherein
p is 0 or 1;
R¹ is selected from the group consisting of C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof;
R² is selected from the group consisting of H, C₁-C₁₀ alkyl, C₂-C₁₀ alkenyl, C₂-C₁₀ alkynyl, C₃-C₁₀ cycloalkyl, C₃-C₁₀ cycloalkenyl, C₃-C₁₀ cycloalkynyl, and combinations thereof; and
R³ is an oxo group.

3. The method of claim 1, wherein p is 1.

4. The method of claim 1, wherein R¹ is C₁-C₁₀ alkyl.

5. The method of claim 1, wherein R² is H.

6. The method claim 1, wherein when p is 1,
R¹ is selected from the group consisting of C₁-C₁₀ alkyl, C₁-C₁₀ alkenyl, C₁-C₁₀ alkynyl, C₃-C₁₀ cycloalkyl, C₃-C₁₀ cycloalkenyl, C₃-C₁₀ cycloalkynyl, and combinations thereof; and
R² is H,
the method further comprises the step of acylating a compound of the general formula (C′), or a stereoisomer thereof to produce a compound of the general formula (E), or a stereoisomer thereof,

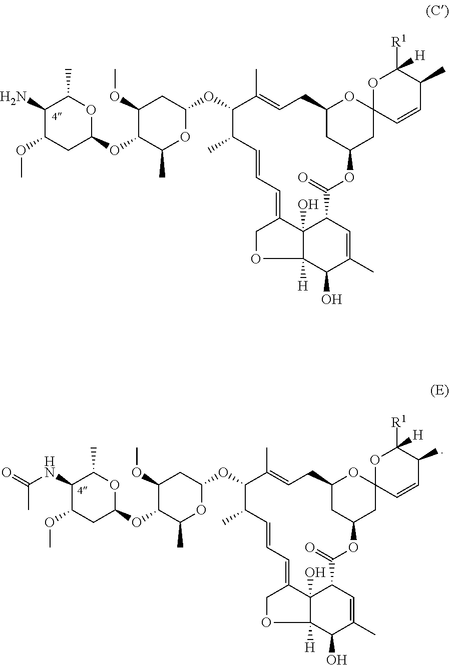

7.-11. (canceled)

12. A pharmaceutical composition comprising a pharmaceutically effective amount of a compound obtained by the method of claim 1 combined with at least one pharmaceutically acceptable excipient.
